(19) United States
(12) Patent Application Publication
(10) Pub. No.: US 2003/0124745 A1
(43) Pub. Date: Jul. 3, 2003

(54) Process for detecting or quantifying a biological reaction using superparamagnetic label

(76) Inventor: Qi Chen, San Gabriel, CA (US)

Correspondence Address: The M.H. Sears Law Firm Chartered, 910 Seventeenth Street, NW, Washington, DC, 20006, US

(21) Appl. No.: 10/298,947

(22) Filed: Nov. 19, 2002

Related U.S. Application Data
(63) Continuation-in-part of application No. 09/978,105, filed on Oct. 17, 2001.
Application No. 09/978,105 is a continuation-in-part of application No. 09/692,463, filed on Oct. 20, 2000, now abandoned.

(51) Int. Cl. G01N 33/553

(52) U.S. Cl. 436/526

(57) ABSTRACT

A method for detecting the occurrence of a biological reaction or quantitating its result employing superparamagnetic particles is disclosed. The particles are first conjugated or adsorbed to identical biomolecules which are members of a biological binding pair and the conjugates or adsorbates so formed are then contacted with a liquid or solid sample known to contain, or suspected of containing, molecules that are the biological binding partners of the biomolecules in the conjugates or adsorbates. The conjugates or adsorbates are contacted with the liquid or solid sample for a time sufficient to enable the formation of a tightly bound, three-dimensional mass comprised of interlinked biomolecules and bound superparamagnetic particles. The mass was exposed to the magnetic field of an experimental instrument for the shortest period necessary to induce magnetization of the superparamagnetic particles, whereupon the magnetic field was immediately removed. It was found that the mass exhibited a measurable nonpermanent magnetization which persisted for at least 20 minutes following exposure to the magnetic field. Experiments were later performed using a SQUID instrument having a constant magnetic field that cannot be switched off during measurements. These experiments confirmed that a measurable non-permanent aggregative magnetization remains in the sample, albeit for a shorter time period. They also showed that the effect persists for a longer period when the sample is kept wet after magnetization than when the sample is allowed to dry thereafter.

This application is a continuation-in-part of pending U.S. application Ser. No. 09/978,105 filed Oct. 17, 2001 which is in turn a continuation-in-part of U.S. application Ser. No. 09/692,463 filed Oct. 20, 2000 and now abandoned.

The present invention relates to a new detection system for recognizing and/or monitoring biological, including biochemical, reactions and especially immunochemical reactions. In this system, the introduction of superparamagnetic force effectively facilitates accurate detection of the degree of such a reaction based upon the concentration, or number, of molecules of one reactant that have participated in the reaction. The invention will find ready application in a variety of in vitro uses, including immunoassays, chromatographic molecular separations, nucleic acid probe analyses, pesticide residue analyses, oligonucleotide probes, and other areas in which biological, including biochemical, reactions are now observed or measured or where such observations and measurements plainly could usefully be made even though not yet reported.

### BACKGROUND OF THE INVENTION

Various references describe the use of certain colloidal metallic particles, and specifically of colloidal gold, as identifiers for unusual cells or molecular entities that may be present in biological tissues and as tags for biological, including biochemical, molecules in a variety of immunoassays, nucleic acid probe analyses, chromatographic separations, oligonucleotide probes and myriads of other specific applications where the monitoring of a biological reaction, the identification of one or more disease-causing organisms, the identification of specific moieties that participate in a particular biological reaction or that may disrupt the normal functioning of such a reaction, the identification of moieties that trigger pathological reactions, and similar information of a biological/biochemical nature is being sought.

Use of colloidal gold to tag these reactions may, with care and the addition of complicated conditions and steps, be conducted in a manner that yields information of a somewhat quantitative nature. Qualitative uses of these colloidal gold tags however, are more easily availed of and, in general, yield more accurate and useful results than attempts to obtain quantitative information with them. To put it another way, colloidal gold in particular offers outstanding advantages as a tag material when the goal is to use it to identify specific biological molecules or moieties, but obtaining reliable quantitative information from assays and other reactions where only colloidal gold tags are used is most often an extremely daunting and time-consuming task.

A number of suggestions have been made for employing magnetic beads or particles as labels for biological reactions, on the premise that magnetic field sensors will yield readings enabling determinations of, e.g., molecular size or yield of desired end product. See, e.g. Adelmann U.S. Pat. No. 5,656,429 and Adelmann, L., J. Assn. for Laboratory Automation 4, No. 3, pp. 32-35 (July 1999).

The Adelmann journal article teaches that off-the-shelf magnetic beads exhibit “remnant [sic] magnetization, i.e., are permanently magnetizable” (p. 33) and that their remanent magnetic field is what enables quantitation and/or detection of polynucleotides and other bound targets. In its examples the beads referred to are of an unusually large size in the order of 800 nm in one instance and 4 microns in another. Such large beads cannot effectively be used as markers for the great bulk of biological reactions because their Theological properties prevent their ready movement through the normally somewhat viscous media in which such reactions occur, and also prevent their ready flow along matrices such as cellulose derivatives, paper, wood, glass etc. upon which such reactions are often performed. The article further teaches that scientists in biomedical and biotechnology laboratories have long recognized a need, when using magnetic beads, for separating excess unbound beads from those that become bound to target whole cells, DNA or proteins—and that this separation is performed by subjecting the mixture to a fixed magnetic field which attracts the unbound beads (Id.) It is noted that both the permanent magnetizability of these beads and the circumstance that a fixed magnetic field attracts the excess beads strongly suggest the beads were ferromagnetic in character and not superparamagnetic.

Baselt U.S. Pat. No. 5,981,297, proposes using magneto-resistive elements, similar to those used for reading magnetic tapes or disks in, e.g. electronic and computer applications, which are described as measuring approximately 20 by 20 μm. (Col. 6 line 34) to detect many particles per element, (as distinguished from a one particle-per-element embodiment also proposed). This magnetoresistive element is first precoated with an insulator and a binding molecule specific to the target molecule is then covalently bound thereto. The thus prepared element is placed in a flow cell to which liquid sample containing target molecule is added, followed by addition of a suspension of 1-5 nm diameter particles that may be ferromagnetic, ferrimagnetic, superparamagnetic or paramagnetic and have a coating of the binding molecule specific to the target molecule. The specification teaches (Col. 7, lines 1-14) that each magnetoresistive element has the purpose to count the particles that bind to the target molecule, and that prior to activating the magnetoresistive detection element, a magnetic device, such as an electromagnet is used to remove non-specifically adhering particles. It says this function is “best provided by sending a brief (˜10-100 ms) pulse of current generated by a capacitative discharge circuit, through an air core electromagnet coil” (Id., lines 11-14). A magnetic field generator (Col. 7, lines 21-38) then magnetizes the bound beads, each of which creates a magnetic field that changes the resistance of the magnetoresistive element to which it is bound and the resistance is then compared to that of a reference element by a Wheatstone bridge, whereupon the data are digitized and conveyed to a microprocessor which determines the total number of beads on the particular magnetoresistive element, from which target molecule concentration can be calculated.

Various other ways of using magnetic or paramagnetic beads to measure a target substance within a sample have also been described. See, e.g. Rapoport U.S. Pat. No. 5,978,694 involving the use of an electrical conductor to measure changes in magnetic susceptibility of a liquid sample when the latter, having present a paramagnetic, diamagnetic or ferromagnetic labelling material (which may be oxygen, which is characterized as “paramagnetic”) that is bound at least in part to a target substance, is subjected to an applied magnetic field.

A German group, Kotitz et al., have demonstrated that ferromagnetic (including ferrimagnetic) nanoparticles can be used to tag various biomolecules. See the Kotitz et al. abstract “Superconducting Quantum Interference Device-Based Magnetic Nanoparticle Relaxation Measurement as a Novel Tool for the Binding Specific Detection of Biological Binding Reactions” J. Appl. Phys. vol. 81. p. 4317 (April 1997) which relates to a conference paper given in November 1996 at the 41st Annual Conference on Magnetism and Magnetic Materials, held at Atlanta, Ga., the related U.S. Pat. No. 6,027,946 which claims a German priority date of Jan. 27, 1995 and names as inventors the same four individuals named as authors on the abstract. Also closely related to the published abstract and the U.S. patent are a further conference paper from the same group published in IEEE Transactions on Applied Superconductivity vol. 7, no. 2, pt.3, pp. 3678-81 (1997) and first given at the 1996 Applied Superconductivity Conference in Pittsburgh, Pa. in August 1996 and a later article from the group published in J. Magnetism and Magnetic Materials vol. 194, no. 1-3, pp. 62-68 in April 1999. In all of these four cited publications the group worked with ferromagnetic, including ferrimagnetic, nanoparticles rather than with superparamagnetic particles. Three of these publications including the U.S. patent, relate to the detection of analytes labelled with these ferromagnetic materials using a detection method the authors (inventors) term “magnetorelaxometric” wherein SQUIDS are used to measure time in milliseconds within which the ferromagnetic particles, after exposure to, and withdrawal of, a magnetic field—both usually done within a magnetically shielded environment—undergo at least a partial reordering of their magnetic moment.

The IEEE Transactions paper, relates to using SQUID to measure remanent magnetization of sample in the absence of an external field. More specifically, this paper describes a process wherein monoclonal antibodies to collagen Type III were coupled to dextrane—coated ferromagnetic iron oxide nanoparticles having an average mean diameter of 13 nm. Meanwhile polystyrene tubes were incubated with collagen type III in PBS, whereby this antigenic material adsorbed onto the tube walls.

The ferromagnetic nanoparticle-labelled monoclonal antibodies in a ferrofluid were added to these prepared tubes and allowed to incubate for 60 minutes. The tubes were each exposed to a magnetic field for 10 seconds. A measurement of magnetic remanence was then made on the ferrofluid—filled tubes, the measurement being performed in a magnetically shielded environment. The tubes were then each decanted and washed three times with PBS and a magnetic remanence measurement was again made on each. The results showed no change in the remanence signals from those obtained while the tubes were still filled with ferrofluid. From this it was concluded that unbound particles, —i.e. particles that did not participate in the antibody-antigen reaction—could be wholly disregarded whether or not they had initially reacted with monoclonal antibody alone to form “blocked particles” and that the measured remanent effect was solely discernible with particle labelled antibodies that bound to the adsorbed antigen on the cell walls. This measured remanent magnetization, moreover, was found to be a linear function of antigen concentration on the tube walls.

The present invention involves the discovery that individual superparamagnetic particles of physical size such that they exhibit average mean diameters between 1 and about 100 nm, preferably between 1 and about 60 nm, and most preferably from 5 nm to 50 nm are not permanently magnetizable (and hence do not possess the remanent magnetization described in the IEEE publication) but nevertheless do acquire, when closely packed together in an interlocked bio-organic matrix, an impermanent magnetization effect that persists long enough to permit informative and highly useful measurements to be made.

### BRIEF DESCRIPTION OF THE INVENTION

The present invention involves using superparamagnetic particles having a physical size as measured by X-ray diffraction and transmission electron microscopy wherein the average mean diameter is in the range from 1 to about 100 nm, preferably 1 to about 60 nm and most preferably between about 5 nm and 50 nm, as labeling agents for at least one selected or suspected reactant of a biological reaction wherein the reactants, upon interaction among or between them, form a three-dimensional mass of tightly packed, often molecularly cross-linked, bioorganic material and bound superparamagnetic (SPM) particles. In such a mass, the SPM particles are closely juxtaposed to one another in all three dimensions of the mass and the result is that, upon subjecting the mass to the magnetizing effect of a magnetic field (e.g., the field exercised by a magnet of about 10,000 Gauss in strength) for a short period in the order of not more than 30 seconds, preferably 10 seconds or less, the closely juxtaposed particles become magnetized.

It has been experimentally shown that this magnetization gradually decays over a period of at least 20-30 minutes and in some cases, longer, to a point where it disappears. The magnetization described in the preceding sentence is referred to herein as “nonpermanent aggregative magnetization” or “residue magnetization”. This non-permanent aggregative magnetization or “residue magnetization” is desirably measured within a set time interval, in minutes, of the withdrawal of the influence of the magnetic field from the mass.

By standardizing this interval to 5 minutes during the work underlying this invention, it was found that a standard curve could be constructed for particles having the same identity characteristics (i.e., particle diameter, surface treatment, and identity of biomolecule to which it is bound) that permits ready calculation of the number or concentration of target molecules that reacted with these particles and were accordingly present in the test sample.

In these experiments it was also found that stray individual particles of superparamagnetic material having a physical size with the same average mean diameter range and having the same surface treatment, whether initially bound to a biomolecule identical to those that actually reacted with the target molecules of the test sample or wholly unbound, did not retain measurable magnetization upon withdrawal of the magnetic field. Because they did not, it was concluded to be unnecessary to perform any step of physically separating them prior to proceeding with measurement of the nonpermanent aggregative magnetization of the agglomerated interacted mass of superparamagnetic particles and bioorganic molecules. The latter produce measurable magnetic readings, it is theorized, because of their lower sensitivity to thermal effects. The unreacted superparamagnetic labelled antibody or other biomolecule has a smaller physical particle size compared to the superparamagnetic-labelled immunocomplex or other superparamagnetic-labelled reacted biomass. The superparamagnetism of the labelled antibody or other biomolecule and the direction of its magnetization are more susceptible to immediate dissipation as a result of thermal effects and, just as in the superparamagnetic particle alone, it is believed that the direction of magnetization in the superparamagnetic-labelled antibody tends to become random very quickly.

Earlier experiments described and discussed herein and in the parent applications were conducted with an experimental prototype measuring instrument, the Ericomp Maglab 2000, which to date has not become available in the marketplace, to the best of applicant's knowledge and information. The present application adds a discussion and description of similiar work performed using a SQUID as the measuring instrument. This work confirms the observations previously made that superparamagnetic particles bound to a conjugate of a biomolecular binding pair, such as an antibody-antigen conjugate, behave differently from both unbound superparamagnetic particles and such particles that have formed a conjugate with a single biomolecule in that the biomolecular pair-bound superpara-magnetic particles will further bind to form a sandwich, e.g., Of tagged antibody-antigen-antibody, and in that form they retain measurable magnetization for a time period thereafter. By contrast, unbound superparamagnetic particles and those bound to a single biomolecule that are in excess or otherwise are incapable of forming a supermagnetic particle-tagged biomolecule-biomolecular binding partner-fixed biomolecule “sandwich” (such as a superparamagnetic particle-tagged antibody-antigen-fixed-antibody“sandwich”), do not retain magnetization and accordingly are readily separated, e.g. by simple washing from the cross-linked structure that the tagged antibody-antigen-fixed antibody sandwiches form in which structure the superparamagnetic particle tags that are present exhibit retained magnetzation over a measurable time period.

It was also found, in the experiments newly described herein employing a SQUID, that in immunochromatographic strips at one end of which tagged antibody-antigen-fixed antibody sandwiches were formed as described hereinafter, the magnetization of the superparamagnetic tags in these sandwich structures persisted longer and at higher intensity if the strip was kept in the wet state than if the strip was first dried and its magnetization then measured. This latter observation, which was tested several times, was unexpected.

### BRIEF DESCRIPTION OF THE DRAWINGS

The attached drawings are as follows:

FIG. 1 is a schematic diagram of a hysteresis curve for typical ferromagnetic material taken from the literature. In the diagram, H represents the applied magnetic field in oersteds and a represents the magnetization resulting therefrom. Magnetization saturation is represented by σₛ while the remanence, or magnetization remaining after removal of the magnetic field is represented by σᵣ. The reverse magnetic field necessary to bring Or to zero is represented by H_{c}, the coercive force.

FIG. 2 is a superimposed plot of the measured hysteresis of superparamagnetic particles coated with small polyacrylic acid and then reacted with a commercially available antibody called bethyl. Three such measurements were made on identically treated particles of three different iron concentrations as measured on particles in suspension—i.e. 2.4 mg/ml. (circles on the plot), 1.0 mg/ml. (squares on the plot) and 0.3 mg/ml. (triangles on the plot). In the plot M represents total magnetization in emu (electromagnetic units) per cc. (“cm³”) while H is the applied magnetic field and in oersteds and Hc is the coercive force in oersteds necessary to impose magnetization (rightward direction from the zero line) or reverse magnetization (leftward from the zero line).

FIG. 2A is a similar plot of superimposed hysteresis measurements made using two different instrumental measuring techniques on polymer coated—(i.e., small polyacrylic acid-coated or chondroitin sulfate A-coated) superparamagnetic Fe₃O₄ particles which particles before coating were identical to those used in the experimental work described herein—and a hysteresis measurement made with a Vibrating Sample Magnetometer (“VSM”) on superparamagnetic particles of Fe₃O₄ conjugated to CPS antibody. In FIG. 2A, the polymer-coated superparamagnetic Fe₃O₄ particle measurements made using a SQUID instrument are represented by solid black squares; the measurements made on polymer-coated superparamagnetic Fe₃O₄ particles are represented by white circles and the measurements of superparamagnetic Fe₃O₄ particle-antibody conjugate are represented by solid black triangles.

FIG. 3 is a plot of measured magnetization in relative magnetic units vs. antigen concentration in ng/ml of two series of superparamagnetic particle-antigen-immobilized antibody sandwiches as measured at the capture line of an ICT device of the type described, e.g., in U.S. application Ser. No. 07/706,639 of Howard Chandler, now U.S. Pat. No. 6,168,956, or any of its various continuing patents and applications, all of which are assigned to Smith Kline Diagnostics, Inc. but exclusively licensed to the assignee of this invention, Binax, Inc. for immunological assays over a wide subject matter area. These data were obtained from work described in Example 3.

FIG. 4 is a plot of Fe concentration, calculated as Fe₃O₄, of the superparamagnetic particle labels used in Example 3 in μg/ml against measured CHW antigen concentration in ng/ml. This data was also obtained from work described in Example 3.

FIG. 5 is a plot of measured magnetization in relative magnetic units against antigen concentration in pg/ml. It also embodies data from work described in Example 3.

FIG. 6 is an X-ray diffraction diagram showing superimposed results of measuring particle size of the superparamagnetic Fe₃O₄ particles used in the experimental work herein described (pattern A) and the same particles after coating with one of the two polymers identified above (pattern B).

FIG. 7 is a plot of M, in electromagnetic units against antigen concentration made with the dry strip data from the readings 1 and 2 (sample series 1 and 2) of Table II, of Example 4.

FIG. 8 is a plot of M in the same units against antigen concentration made using the wet strip data from the reading 1 series of Table IV, Example 4.

FIG. 9 is a similar plot to FIG. 7 made from the dry strip data of the reading 1 and 2 series of samples of Table IIA, Example 5.

FIG. 10 is a similar plot to FIG. 8, constructed from the wet strip date of the reading 1 series of samples of Table IVA of Example 5.

### DETAILED DESCRIPTION OF THE INVENTION

According to the present invention, superparamagnetic particles which are individually too small to maintain any degree of remanent magnetization after exposure to the action of a magnetic field of the strength of about 10,000 Gauss for a period as short as possible, preferably 10 seconds or less, and not more than 30 seconds, have been shown to acquire measurable non-permanent aggregative magnetization—i.e., collective magnetization of an aggregated, interacted three dimensional biomass—when closely incorporated into a tightly packed three-dimensional mass with agglomerated biological material such as, e.g., a mass of labeled superparamagnetic antibody-antigen-immobilized antibody “sandwiches”, a clotted mass of labelled blood platelets, a mass of chromatographically separated protein, and the like.

Use of superparamagnetic particles as labels for biological, including biochemical, reactions offers substantial advantages over many of the labels now used, e.g., in various assay systems. For example, superparamagnetic particles, in contrast to ferromagnetic particles, do not display remanent magnetization and have no magnetic properties until subjected to the influence of a magnetic field. They are accordingly virtually unlimitedly shelf-stable in contrast to many of the labelling materials in common use, including colloidal metals, enzymes, chemiluminescent agents, radioactive tracers etc.

Their stability renders them easy to mix with other substances, to suspend freely in liquids and otherwise to work with, so long as they are not exposed to magnetic fields of sufficient intensity to excite magnetization.

Nonpermanent aggregative magnetization as observed in the context of the present invention is a measurable phenomenon which is a straight line function of the concentration, or number, of target biomolecules in a test sample. However, care must be taken to measure the nonpermanent aggregative magnetization at the same time interval after removal of the magnetic field that induces this magnetization if one is to achieve comparable results in a series of tests—e.g., tests conducted at different concentrations of a target analyte molecule, tests undertaken to construct a standard curve, tests undertaken with the intent to rely on an already constructed standard curve to determine concentration present in a sample, etc.

It is believed that particle size, surface features of the particles, magnetic field strength and time employed in the magnetization step, as well as the mean distance between the magnetized particles trapped in the end product mass of bioorganic material and bound particles, will all play a role in the length of time within which nonpermanent aggregative magnetization persists and the rate at which it decays. It also appears that the decay, at least in systems so far tested, occurs at a rate such that correlation of the number of bound particles with the concentration of a target analyte or other target molecule can be achieved when total magnetization measurements are made not only at the 5-minute interval following the magnetization step that was chosen for Example 3 and other work wherein the Ericomp instrument was utilized, but at some other uniform time interval from the magnetization step, such as the shorter period chosen in work involving the SQUID instrument. Care must be taken, of course, that the measurements are taken at an interval such that measurement of total magnetization yields readings that are in excess of the reading for any background magnetization that may need to be deducted, depending upon the “platform” or biological matrix that may be present. Furthermore, before selecting a different interval for measurement of total magnetization, one needs to ensure that the rate of decay of non-permanent aggregative magnetization follows a consistent pattern for superparamagnetic particles that have the same treatment history.

As used herein, “superparamagnetic particles” refers to particles that are magnetizable but retain no remanent magnetization when tightly packed together in close association in a mass of inter-reacted bio-organic materials and that, when measured individually after attempted magnetization, exhibit no remanent magnetization. These particles may comprise pure metals such as Fe, Co, Cd, Zn, Mg, Mn, etc. that are known to be readily magnetizable, iron oxide, CoFe₂O₄, MgFe₂O₄ and oxides of other metals that are known readily to be magnetizable when a mass thereof is subjected to the influence of a magnetic field, such as iron, zinc, nickel, cadmium, cobalt, etc. Also usable are, e.g. Fe and its oxides and other metal combinations, and oxides thereof, that exhibit spinel structure upon examination by X-ray diffraction and Transmission Electron Microscopy. It should be noted that pure metals are superparamagnetic only within a physical size range wherein the average mean diameters are confined with a few nanometers. Superparamagnetic particles of pure metals are also chemically unstable whereas oxides of corresponding superparamagnetic particles are relatively inert and maintain their superparamagnetism within a broader physical size range.

These superparamagnetic particles are not intrinsically reactive with bio-organic materials and often are desirably coated with a substance that enables them to react with a binding partner of the target molecule which is to be monitored, assayed for, or otherwise located and quantitated. Various methods of and materials for such coating are known and have been used in the past for coating polymers or glass, including glass beads and solid polymer—comprising inserts or “dipsticks” that have been used in various assay systems. The same coating methods and materials are useful in coating superparamagnetic particles to be used in detecting end products of biological, including biochemical, reactions. Various methods of adsorption are also well known wherein proteins and the like are directly adsorbed on, e.g. iron oxides and the like and they also may be utilized in this invention to improve the reactivity of the particles.

Superparamagnetic particles are distinguished from both ferromagnetic, including particles, which acquire permanent remanent magnetization upon exposure to an external magnetic field, and also from paramagnetic materials, which have a positive magnetic susceptibility less than 0.001 times that of ferromagnetic materials. The magnetic susceptibility of superparamagnetic materials lies between that of ferromagnetic materials and paramagnetic materials and hence that of the superparamagnetic particles and particles is referred to herein intermediate that of ferromagnetic and paramagnetic particles. The superparamagnetic systems, exhibit a slow relaxation time—i.e., they revert from a magnetized state to a non-magnetized state slowly. The core behavior superparamagnetic relaxation time is the average time it takes particle relaxation to jump from one direction to another. The particles used in the work underlying this application were of 5-15 nm average mean diameter as measured by X-ray diffraction and Transmission Electron Microscopy. They were of pure Fe₃O₄ having spinel structure, as confirmed by X-ray diffraction and transmitted electron microscopy.

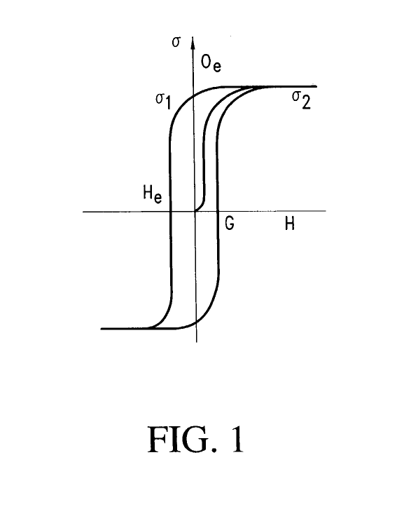

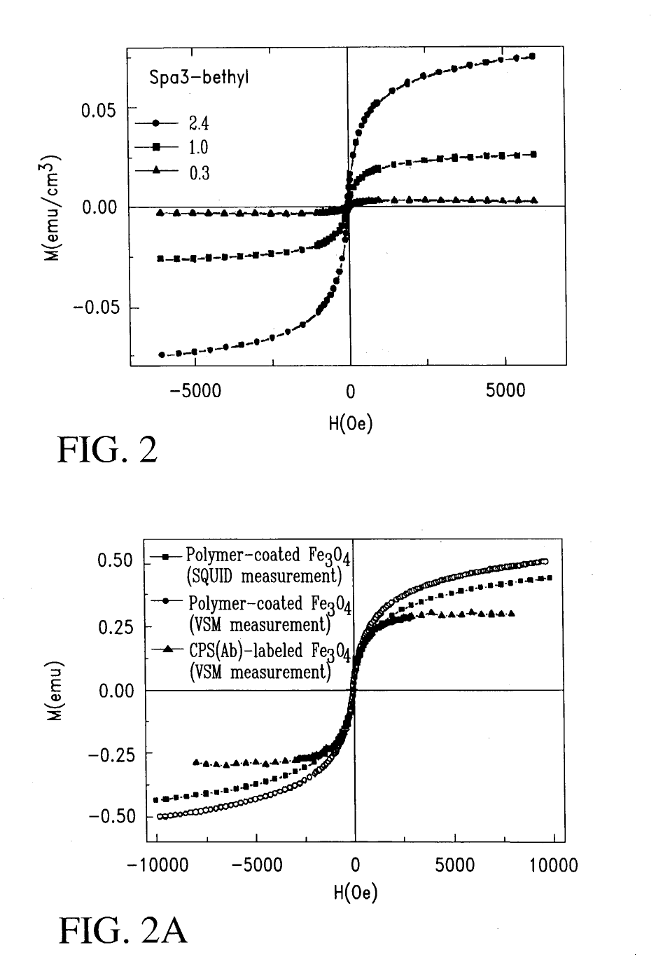

Superparamagnetic materials are known by physicists not to exhibit remanent magnetization. The hysteresis loop of superparamagnetic materials (i.e., the plot obtained by plotting magnetization against magnetic field strength) is curve-like and it typically resembles those shown in FIGS. 2 and 2A hereof. This is in contrast to the typical hysteresis loop of ferromagnetic materials (FIG. 1) and the linear hysteresis plot obtained with paramagnetic materials. Most usually, superparamagnetic particles have a small average mean diameter in the order of less than 50 nm, often 30 nm or less, in physical size as measured by X-ray diffraction and Transmission Electron Microscopy—although in some systems larger sizes of particles with superparamagnetic properties have been observed. (It is noted parenthetically that both their size and behavior after removal from a magnetic field suggest that the 800 nm and larger particles referred to in the Adelmann et al. article were ferromagnetic and not superparamagnetic). Still further, it is typical of superparamagnetic particles that the magnetization they may exhibit when subjected to a magnetic field, decays with time until it dissipates altogether. Finally, superparamagnetic particles possess a degree of magnetic ordering—i.e., they have what is called a subdomain structure consisting of clusters of varying sizes containing some atoms with unpaired electrons in the unmagnetized state, but the clusters are small and scattered in comparison to the “domain structure” of ferromagnetic materials which are characterized by larger clusters called domains in which each atom or other structural unit has unpaired electrons that impart a net magnetic moment. In these latter materials, each domain exhibits a directional magnetic effect which is the vector sum of all unpaired electrons present in that domain.

In sum, ferromagnetic materials have strong magnetic ordering, superparamagnetic materials have some magnetic ordering, but much less than ferromagnetic materials. The presence of magnetic ordering in superparamagnetic materials has been confirmed by neutron diffraction measurements. See Chen et al., “Synthesis of Superparamagnetic MgFe₂O₄ Nanoparticles by Coprecipitation”, J. Magnetism and Magnetic Materials, vol. 94, pp. 1-7 (1999). Paramagnetic materials have no magnetic ordering.

For a good technical description of similarities and differences physicists recognize among “ferromagnetic”, “superparamagnetic”, and “paramagnetic” materials, see Chen et al., “Size-dependent Superparamagnetic Properties of Mg₂FeO₄ Spinel Ferrite Nanocrystallites”, Appl. Phys. Letters, vol. 73, pp. 3156-8 (1998).

It is possible that the ability to measure non-permanent aggregative magnetization in agglomerated three-dimensional reaction products comprising bio-organic materials in close association with superparamagnetic particles is attributable to the relatively stable macrostructure of these reaction products, which macrostructure holds the incorporated particles in place and prolongs the decay of the magnetization imparted by exposure to a magnetic field. Applicant, however, has not established this or any other scientific explanation for the reproducible phenomenon observed in the experimental work relating to this invention and hence do not intend to be bound to any particular explanation.

The present invention provides enhancements in a virtually unlimited spectrum of in vitro biological reactions including at least immunoassays, DNA probes, oligonucleotide probes, chromatographic molecular separations and other biological reactions where it is desirable to quantitate the amount of a target molecule present. It is believed that this invention may also be useful in monitoring certain in vivo biological reactions. The detection system of this invention is beneficially employed in immunoassays generally, but especially in immunochromatographic and other “lateral flow” assays and in so-called “flow through” assays—i.e., those involving vertical flow steps in which the reactants are brought together.

The Midwest Scientific Co. newsletter, Shark Bytes, for October 2000 describes a form of assay now in development at Ohio State University wherein a compact disc (“CD”) rotated by a compact disc player is equipped with tiny reservoirs and channels that cause medical samples suspected of containing target analyte to mix with tiny pools of test reagents. Including superparamagnetic particle tagged binding partners for analytes suspected of being present in tiny pools on such test platforms would lead to very useful assays capable of being rapidly performed and rapidly evaluated via the computer anticipated to be included in the CD player of the Ohio State system. This computer could readily be programmed to read non-permanent aggregative magnetization imparted by an external magnetic field in digital form and to correlate this reading to stored information corresponding to a standard curve. As those skilled in immunoassays will recognize, one CD with associated CD player-computer combination could readily be adapted to perform several assays simultaneously on portions of a single test sample by providing, e.g., different antibodies for different target analytes conjugated to superparamagnetic particles placed in different “pool” regions of the CD.

In addition to CD's, other “platform” materials upon which superparamagnetic particle-tagged biomolecules may be reacted with target biomolecules in a test sample are contemplated to be useful in work performed within the scope of this invention. Possibilities specifically explored in preliminary work, in addition to what the specific examples below show, are balsa wood and glass. With balsa wood, it was found that even though the material is intrinsically non-magnetic and non-magnetizable its capillarity may lead to readings of non-permanent aggregative magnetization that exhibit a very large standard deviation. It is believed that filling these capillaries with non-magnetizable plastic or with a substance such as bovine serum albumin, other proteins, polyethyleneglycol or other substances well known for blocking capillarity in “dipstick” type immunoassay devices described in the prior art would render balsa wood more acceptable as a platform. Glass was found to avoid the capillarity problem and to be a satisfactory platform material, provided that appropriate background readings are obtained, allowing one to compensate for the fact that most glass slides contain sufficient iron to be magnetizable to a low degree upon exposure to a magnetic field. This makes it necessary to determine the background signal and subtract it from sample readings whenever biological reactions wherein one reactant is labelled with superparamagnetic particles according to this invention are run on glass as a platform.

To measure non-permanent aggregative magnetization, various instruments may be used. In this regard, several different research and development groups are in process of developing relatively low cost measuring instruments which apply knowledge gleaned from high resolution magnetic recording technology and computer disk drive technology. One of these is the Ericomp Maglab 2000, at least one early prototype version of which is illustrated in the Adelman J. Assn. for Lab. Automation article cited hereinabove. Another is the Quantum Design, Inc. instrument described in U.S. Pat. No. 6,046,585 issued Apr. 4, 2000. Another Quantum Design, Inc. instrument is described in WO 01/40790 published Jun. 7, 2001.

The following specific examples illustrate the substitution of superparamagnetic labelling according to this invention for colloidal gold in an immunochromatographic (“ICT”) assay for Canine Heart Worm that is commercially available from Binax, Inc, assignee of this patent application.

### EXAMPLE 1

### Selection of Coating Agent for Superparamagnetic Particles

Superparamagnetic particles can be coated with a reagent that has free carboxyl functional groups in order to be capable of covalent coupling to a particular antibody, such as the antibody to Canine Heart Worm (“CHW”). Initial work was accordingly performed to ascertain the coating material of choice for this purpose.

Because rheological properties are important to the successful operation of ICT assays and past experience with colloidal gold labels has shown smaller particles to be rheologically superior, 10 nm diameter Fe₃O₄ particles were chosen for this work. Their size was confirmed by X-ray diffraction and by Transmission Electron Microscopy.

Two polymeric coating materials were tested on separate lots of superparamagnetic particles using the coating method described in U.S. Pat. No. 5,547,682. One lot of these particles was coated with Chondroitin Sulfate A (“CSA”) and the other with small polyacrylic acid (“sPAA”). The coated particles in suspension, in each instance, had a diameter of 40 to 60 nm, as determined by dynamic light scattering. Both the CSA—coated and the sPAA coated superparamagnetic particles were further tested and it was shown thereby that CSA—coated particles were superior in stability and ability to bind to antibodies. The hysteresis loop as measured by Vibrating Sample Magnetometer (“VSM”) tests for three sets of particles having varying iron concentrations, all of which were sPAA coated and had the commercially available antibody bethyl bonded thereto is shown in FIG. 2. These particles in uncoated form are identical to those which were the starting materials for Example 3. FIG. 2 shows the typical hysteresis curve shape of superparamagnetic materials and also confirms that these particles had no remanent magnetization. FIG. 2A shows hysteresis measurements made with coated particles of superparamagnetic 10 nm Fe₃O₄ with a SQUID instrument (curve with black squares) and by VSM (curve with white circles). Also, shown on FIG. 2A is a plot of hysteresis measurements made by VSM of the same coated particles to which a carboxy polysaccharide antibody was conjugated. All three again exhibit the typical shape of superparamagnetic material hysteresis behavior and confirm the particles lack of remanent magnetization.

### EXAMPLE 2

### Preparation of Superparamagnetic Particle-Labelled Antibodies

CSA-coated superparamagnetic particles prepared as in Example 1 were covalently coupled to anti-Canine Heart Worm (“CHW”) antibody identical to the anti-CHW antibody used in the manufacture of the commercially available Binax ICT test for CHW antigen and were then suspended in phosphate-buffered saline solution containing 10 mg/ml of bovine serum albumin pending their use as in Example 3.

### EXAMPLE 3

### Performance of ICT Assay for CHW Using Superparamagnetic Labels

ICT flow path test strips of nitrocellulose were treated in the same manner as those used in the Binax commercially available ICT assay for CHW. These strips were incorporated into dipstick-type devices by the lamination of absorbent pad components overlapping opposite ends of the flow path test strip. A series of solutions containing CHW antigen were prepared with antigen concentrations ranging from 100 pg/ml to 200 ng/ml. 190 μl of each of these antigen solutions were dispensed into separate wells of a new polystyrene 96 well microtiter plate. To these samples were added 5 μl of the CSA-coated superparamagnetic particle-labelled anti-CHW antibodies of example 2. Labeled antibody/antigen solution mixtures were incubated for 15 minutes at room temperature. The sample receiving end of a dipstick device was added to each labeled antibody/antigen solution mixture immediately following this incubation, causing the mixture to flow into the capture zone. Immobilized unlabelled rabbit polyclonal anti-CHW antibodies bound to the strip in the capture zone thereupon reacted with antigen-superparamagnetic labelled antibody conjugates to form immobilized antibody-antigen-superparamagnetic labelled antibody “sandwiches” along the capture line. The ICT strips were removed from the ICT devices after 15 minutes, and exposed to a magnetic field of 10,000 Gauss for 10 seconds each. After 5 minutes from the removal of the magnetic field, each strip was placed in an Ericomp Maglab 2000 instrument with the capture line in the field of view of the detector and its non-permanent aggregative magnetization was read.

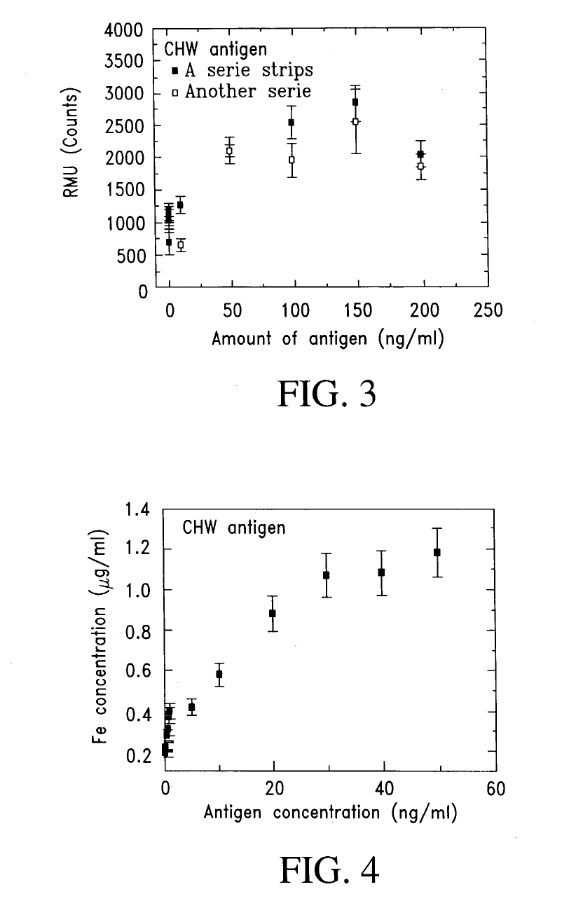

Each antigen solution was tested in duplicate in the ICT test as described. The readings of non-permanent aggregative magnetization for both series of sample having known antigen concentrations above 1 ng/ml have been graphed in FIG. 3 against antigen concentration. The Fe content of the immune complexes at the capture lines of ICT devices used in the duplicate series of tests was determined by a chemical calorimetric procedure using a commercial ferrizine test from Sigma Chemical Co. It was found that the calorimetric chemical test results and the magnetization readings correlated well, as shown in FIG. 4, a plot of iron concentration calculated as Fe₃O₄, in μg/ml, against antigen concentration in ng/ml.

In the ICT tests as performed in this example, any labelled antibody initially deposited at the flow path threshold that fails to react with antigen in the sample flows past the capture zone and into another pad positioned upstream from that zone. These unreacted paramagnetic particle-labelled antibodies were subjected to the effect of a magnetic field of 10,000 Gauss for 10 seconds, set aside for 5 minutes and then placed in the sensor area of the Ericomp Maglab 2000 instrument and found to exhibit no measurable magnetization.

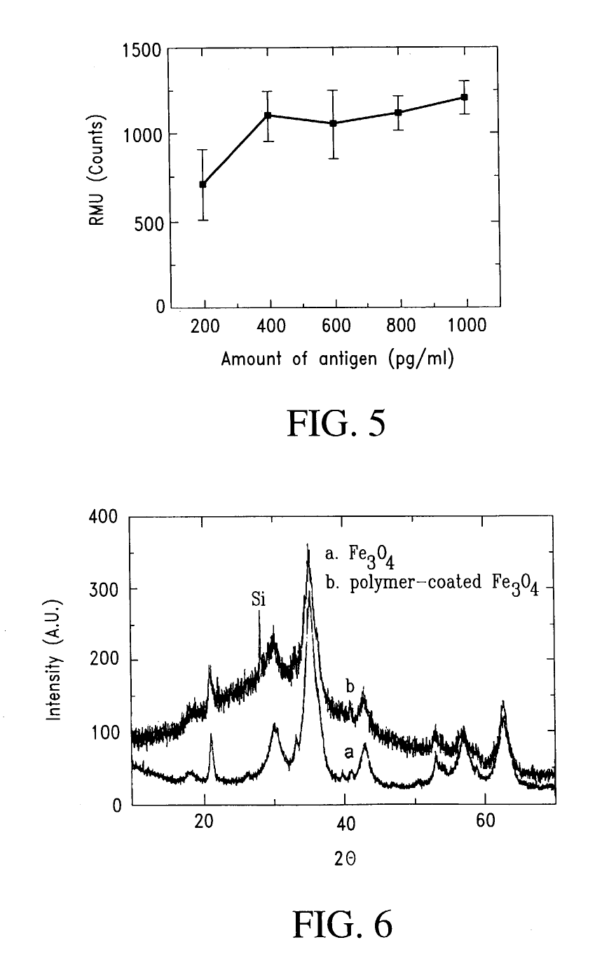

From the results of the foregoing examples, it was determined that the relationship between magnetic reading in relative magnetic units (“RMU”) and concentration of antigen (the target analyte) is an essentially linear function in the range of 1 ng antigen/ml to 150 ng antigen/ml. See FIG. 3, a plot of relative magnetic units against antigen concentration in ng per ml for each of the two series of CHW assays performed. The instrument noise, however, caused large standard deviations in the readings of samples having concentrations of antigen below 1 ng/ml. This is illustrated in FIG. 5, a plot of measured values in relative magnetic units against antigen concentration in pg per ml. The fact that ICT strips having 200 pg/ml of added antigen had non-permanent aggregative magnetization that could be read when that antigen was incorporated in labelled antibody-antigen-immobilized antibody sandwiches collected in a mass, subjected to 10,000 Gauss of magnetic field for 10 seconds, and then set aside for five minutes is an indication nonetheless that the sensitivity of the test is significantly enhanced by substituting superparamagnetic labels for colloidal gold labels.

With an improved instrument having reduced noise, it is clear that the physical sensitivity of superparamagnetic labelling as described approaches 0.1 ng of Fe calculated as Fe₃O₄ or about 10⁻¹⁸ per mole, while the broad dynamic sensitivity range will fall between about 1 and 10⁶ relative units and has potentially high tolerance to interference from various biological matrices that may present.

### EXAMPLE 4

Dipstick-type devices as described in the first three sentences of Example 3 were prepared. Solutions respectively containing 100 ng/ml., 150 ng/ml and 200 ng/ml of the canine heartworm antigen, which is the target antigen of the commercially available Binax assay for canine heartworm, were prepared and dispensed into separate wells of a new polystyrene microtiter plate. To each sample was added 5 μl of CSA-coated superparamagnetic-particle labeled anti-CHW antibodies as described in Example 2. Each of the labeled antibody/antigen mixtures resulting therefrom was incubated for 15 minutes at room temperature, whereupon the sample-receiving end of a dipstick device was added to each well causing the mixture to chromatograph along the dipstick strip to its capture zone where “sandwiches” of superparamagnetic labeled antibody-antigen-immobilized antibody composition formed along the capture line. After 15 minutes, the dipstick devices were removed from the wells and a segment of each ICT strip, encompassing all of the capture line of that dipstick device, was cut out, allowed to dry and exposed to a magnetic field of 1 Tesla ('T”) within a SQUID (i.e. 10,0000 oersteds, also referred to as “10,000 Gauss”) for 10 seconds. The magnetic field was then turned off, and after 3½ minutes, a measurement of the magnetic signal of each segment was made. The premise on which the experiment was based was that the applied magnetic field aligns the magnetic moment of all of the superparamagnetic nanoparticles in the direction of the magnetic field, including those particles bound to the antibody-antigen-fixed antibody “sandwich” formed at the capture line and those not so bound. When the field is turned off, the particles not bound into the “sandwich” revert rapidly to their original nonaligned state, whereas those bound into the interlinked structure of the sandwich are slower to revert. The 5.0 minute time interval to reading corresponds to the time lag to measurement in the experiments with the Ericomp instrument.

The results of these experiments are shown in Table I:

TABLE I

| | Antigen concn. of sample: | | | |
|---|---|---|---|---|
| | 0 ng./ml | 100 ng./ml | 150 ng./ml | 200 ng./ml |
| Magnetic | 6.95 × 10⁻⁷ | 7.12 × 10⁻⁷ | 8.35 × 10⁻⁷ | 9.19 × 10⁻⁷ |
| Reading after | | | | |
| 5 min. | | | | |

The “magnetic reading” is in electromagnetic units (emu).

Another series of experiments was run in precisely the same manner, except that the first reading was taken at 3.5 min., which is the time needed for the magnetic field of the SQUID to revert from 1 Tesla to 0, followed by a second reading at 4.0 min and a third at 4.5 minutes. The results are shown in Table II, where blanks indicate that no reading was recorded.

TABLE II

| | Antigen concn. of sample/ | | | |
|---|---|---|---|---|
| | 0 ng./ml | 100 ng./ml | 150 ng./ml | 200 ng./ml |
| reading #1 | 6.90 × 10⁻⁷ | 7.11 × 10⁻⁷ | 8.30 × 10⁻⁷ | 9.20 × 10⁻⁷ |
| reading #2 | 8.12 × 10⁻⁷ | 9.40 × 10⁻⁷ | 9.04 × 10⁻⁷ | 9.47 × 10⁻⁷ |
| reading #3 | 8.5 × 10⁻⁷ | 7.71 × 10⁻⁷ | | 9.9 × 10⁻⁷ |

Table III shows results of another series of experiments in which the antigen levels in the initial solutions were, respectively 50 ng/ml, 100 ng/ml and 200 g./ml. In all other respects this series of experiments was conducted just like the experiments, results of which appear in Table II.

TABLE III

| | Antigen conc. of sample: | | | |
|---|---|---|---|---|
| | 0 ng/ml | 50 ng./ml | 100 ng./ml | 200 ng./ml |
| reading #1 | | 8.87 × 10⁻⁷ | 7.90 × 10⁻⁷ | 9.66 × 10⁻⁷ |
| reading #2 | 8.58 × 10⁻⁷ | 8.23 × 10⁻⁷ | 7.47 × 10⁻⁷ | 1.00 × 10⁻⁶ |
| reading #3 | 8.48 × 10⁻⁷ | 8.18 × 10⁻⁷ | 7.54 × 10⁻⁷ | 1.00 × 10⁻⁶ |

A further series of experiments was performed in the same manner as those on which the readings in Tables II and III were obtained with the exceptions that (1) the solution antigen levels were, respectively 1 ng/ml, 10 ng/ml. and 200 ng/ml. and (2) the readings were taken on wet, rather than dry, segments. The results are shown in Table IV.

TABLE IV

| | Antigen conc. (In ng/ml) | | | |
|---|---|---|---|---|
| | 0 ng./ml | 1 ng./ml | 10 ng./ml | 200 ng./ml |
| reading #1 | 1.25 × 10⁻⁸ | 1.82 × 10⁻⁶ | 1.71 × 10⁻⁶ | 1.79 × 10⁻⁶ |
| reading #2 | | | 1.49 × 10⁻⁶ | |

### EXAMPLE 5

Because many instruments currently available operate only with a constant magnetic field which is initially applied to magnetize the superparamagnetic particles present and then must remain during measurement, it was decided to run parallel experiments in the same manner as those shown in each of Tables I, II, III and IV of Example 4 wherein the SQUID was maintained under a constant magnetic field of 1 Tesla throughout. The following Table IA shows readings obtained under a constant field of ITesla on samples identically prepared to the samples of corresponding antigen concentration in Table I. These samples, like those for which readings in Table I are shown were measured at 5.0 minutes after the initial 10 second exposure of each sample to the magnetic field:

TABLE IA

| | Antigen conc. in ng./ml | | | |
|---|---|---|---|---|
| | 0 ng./ml | 100 ng./ml | 150 ng./ml | 200 ng./ml |
| Measurement | 1.24 × 10⁻⁴ | 7.65 × 10⁻⁵ | 9.13 × 10⁻⁵ | 4.47 × 10⁻⁵ |
| in electro- | | | | |
| magnetic units | | | | |

Table IIA shows readings obtained on samples prepared identically to those in Table II wherein a constant magnetic field was maintained throughout. In this table, reading #1 was taken 3.5 minutes after the initial 10 second exposure of each sample to the constant magnetic field while reading #2 was taken at 4.0 minutes and reading #3 at 4.5 minutes.

TABLE IIA

| | Antigen conc. | | | |
|---|---|---|---|---|
| | 0 ng./ml | 100 ng./ml | 150 ng./ml | 200 ng./ml |
| reading #1 | 1.25 × 10⁻⁴ | 7.65 × 10⁻⁵ | 9.13 × 10⁻⁵ | 4.5 × 10⁻⁵ |
| reading #2 | 6.98 × 10⁻⁵ | 0.19 × 10⁻⁵ | 9.81 × 10⁻⁵ | 7.28 × 10⁻⁵ |
| reading #3 | 9.98 × 10⁻⁵ | 1.02 × 10⁻⁴ | | 6.24 × 10⁻⁵ |

Table IIIA shows readings obtained on samples prepared identically to those in Table III with readings taken under the same magnetic field and at the intervals specified for Table IIA. Ant

| | Antigen conc. | | | |
|---|---|---|---|---|
| | 0 ng/ml | 50 ng/ml | 100 ng/ml | 200 ng/ml |
| reading #1 | 9.88 × 10⁻⁵ | 5.80 × 10⁻⁵ | 6.16 × 10⁻⁵ | 6.43 × 10⁻⁵ |
| reading #2 | 1.00 × 10⁻⁴ | 5.84 × 10⁻⁵ | 6.24 × 10⁻⁵ | 6.54 × 10⁻⁵ |
| reading #3 | 9.97 × 10⁻⁵ | 6.20 × 10⁻⁵ | 6.70 × 10⁻⁵ | 6.37 × 10⁻⁵ |

Table IVA shows readings taken under the same magnetic field on wet segments prepared in exactly the same way as the corresponding samples on which the readings of Table IV were obtained.

| | Antigen conc. | | | |
|---|---|---|---|---|
| | 0 ng/ml | 1 ng/ml | 10 ng/ml | 200 ng/ml |
| reading #1 | 4.07 × 10⁻⁵ | 1.28 × 10⁻⁴ | 1.41 × 10⁻⁴ | |
| reading #2 | 4.05 × 10⁻⁵ | 1.26 × 10⁻⁴ | 1.391 × 10⁻⁴ | |
| reading # | 4.04 × 10⁻⁵ | 1.26 × 10⁻⁴ | 1.388 × 10⁻⁵ | 1.66 × 10⁻⁴ |

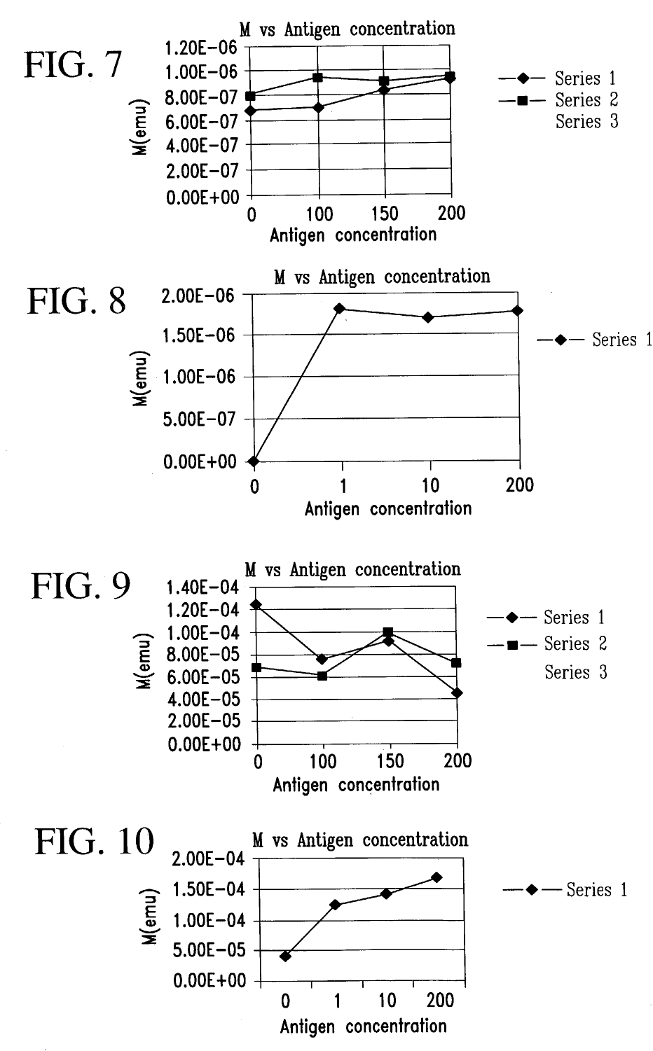

In the various data shown in Examples 4, the readings of Tables IV taken on wet segments, each embracing the capture zone of an ICT strip, show that the nonpermanent aggregative magnetization is retained to a greater degree of intensity for a longer period of time and with a more pronounced dose dependence in these “wet segment” or “wet strip” samples than it is in the corresponding dried samples, at least when the measurement is made with a SQUID instrument. This is more easily seen by comparing FIGS. 7 and 8, where FIG. 7 is a graph of M—i.e., reading in electromagnetic units—against antigen concentration of the data for dry strip readings #1 and #2 in Table I and FIG. 8 is a similar graph of the data for wet strip reading #1 of Table IV.

Surprisingly, the data of Example 5 where readings were taken in the SQUID under a constant field, also show a much more pronounced dose dependent response for “wet segment” or “wet strip” samples than in the corresponding dried samples. Again, this can be easily seen in FIG. 9, a graph of M, as defined above, against antigen concentration for the dry strip readings of Table IA; readings #1 and #2 as compared to FIG. 10, a similar graph of the wet strip reading from Table IVA.

These findings indicate that superior performance, specifically as relates to minimizing signal to noise ratios and hence maximizing sensitivity, can be obtained when samples to be tested are maintained in a wet state. This phenomenon pertains not only to the nonpermanent aggregative magnetization measured followed magnetization and measurement subsequent to removal of the field, but also in the more common measurement systems that employ a constant magnetic field during measurement.

While the invention has been exemplified in the context of a well known immunodiagnostic system specific to the antigen of the causative agent for the canine disease Dirofilaria immitis, the vast range of applications in which it will produce greatly improved results or will enable precise quantitative measurement of observed phenomena previously deemed to be difficult to impossible to measure will be readily apparent to those ordinarily skilled in immunochemistry and/or biology. It is accordingly intended that the scope of this invention be limited only to the extent of the scope of the appended claims.

We claim:

1. A process for detecting a biological reaction which comprises:
(a) conjugating or adsorbing to each of a group of superparamagnetic particles identical biomolecules which are members of a biological binding pair,
(b) contacting the product of step (a) with a sample selected from among liquids and solids, containing or suspected of containing molecules which comprise the biological binding partner of the biomolecules conjugated to or adsorbed on the superparamagnetic particles,
(c) permitting the superparamagnetic particle-biomolecule conjugates or superparamagnetic particle: biomolecule adsorbates from step (a) to react with any biological binding partner molecules present in the aforementioned sample to form a complex, tightly bound, three-dimensional mass comprising interlinked biomolecules and bound superparamagnetic particles;
(d) exposing the said mass to a magnetic field for the shortest period necessary to induce magnetization of the superparamagnetic particles in said mass and then immediately removing the magnetic field, whereupon the superparamagnetic particles in said mass exhibit in concert measurable nonpermanent aggregative magnetization which persists for a period of at least 20 minutes following exposure to the magnetic field, and either
(e) confirming the presence of such magnetization with a suitable instrument if only a qualitative result is desired, or
(f) measuring the intensity of the magnetic signal of the said nonpermanent aggregative magnetization before it dissipates and correlating it to the quantitative concentration, or number, of one of the biomolecules of step (a) or step (b) that participated in forming the mass referred to in step (c).

2. A process according to claim 1 in which the superparamagnetic particles comprise Fe₃O₄ particles having an average mean diameter as measured by X-ray diffraction and Transmission Electron Microscopy of 1 nm to about 100 nm.

3. A process according to claim 2 in which each superparamagnetic particle is conjugated to an antibody, the sample in step (b) is a liquid sample which contains the antigen that is the specific binding partner of said antibody, and a quantitative result is obtained by performing step (f).

4. A process according to claim 3 in which the period of exposure to a magnetic field in step (d) is 5-10 seconds and the average mean diameter of the superparamagnetic particles as determined by X-ray diffraction and Transmission Electron Microscopy is in the range from 5 nm to 60 mn.

5. A process according to claim (4) which is an immunoassay in which the antigen content of the sample is quantified in step (f).

6. A process according to claim 5 which is conducted in lateral flow format.

7. A process according to claim 6 which is conducted in the format of an immunochromatographic assay.

8. A process according to claim 5 which is conducted in a vertical flow or flow-through format.

9. A process according to claim 1 in which the superparamagnetic particles comprise those selected from among superparamagnetic particles of a single magnetizable metal or superparamagnetic particles of two combined magnetizable metals or superparamagnetic particles of oxides of either a single magnetizable metal or two combined magnetizable metals.

10. A process according to claim 9 in which each superparamagnetic particle is conjugated to an antibody, the sample in step (b) is a liquid sample which contains the antigen that is the specific binding partner of said antibody, and a quantitative result is obtained by performing step (f).

11. A process according to claim 10 in which the period of exposure to a magnetic field in step (d) is 5-10 seconds and the average mean diameter of the superparamagnetic particles as determined by X-ray diffraction and Transmission Electron Microscopy is in the range from 5 nm to 50 nm.

12. A process according to claim 11 which is an immunoassay in which the antigen content of the sample is quantified in step (f).

13. A process according to claim 12 which is conducted in lateral flow format.

14. A process according to claim 13 which is conducted in the format of an immunochromatographic assay.

15. A process according to claim 12 which is conducted in a vertical flow or flow through format.

16. A process according to claim 9 in which the superparamagnetic particles comprise superparamagnetic particles of an oxide of two combined magnetizable metals, which particles exhibit a spinel structure as determined by X-ray diffraction analysis and Transmission Electron Microscopy.

17. A process according to claim 16 in which each superparamagnetic particle is conjugated to an antibody, the sample in step (b) is a liquid sample which contains the antigen that is the specific binding partner of said antibody, and a quantitative result is obtained by performing step (f).

18. A process according to claim 17 in which the period of exposure to a magnetic field in step (d) is 5-10 seconds and the average mean diameter of the superparamagnetic particles as determined by X-ray diffraction and Transmission Electron Microscopy is in the range from 5 nm to 50 nm.

19. A process according to claim 18 which is an immunoassay in which the antigen content of the sample is quantified in step (f).

20. A process according to claim 19 which is conducted in lateral flow format.

21. A process according to claim 20 which is conducted in the format of an immunochromatographic assay.

22. A process according to claim 19 which is conducted in a vertical flow or flow through format.

23. A process according to claim 2 in which identical biomolecules are adsorbed to superparamagnetic particles in step (a).

24. A process according to claim 23 in which the period of exposure to a magnetic field in step (d) is 5-10 seconds and the average mean diameter of the superparamagnetic particles as determined by X-ray diffraction and Transmission Electron Microscopy is in the range of 5 nm to 50 nm.

25. A process according to claim 24 which is an immunoassay.

26. A process according to claim 25 which is conducted in lateral flow format.

27. A process according to claim 26 which is conducted in the format of an immunochromatographic assay.

28. A process according to claim 26 which is conducted in a vertical flow or flow-through format.

29. A process according to claim 23 in which the superparamagnetic particles comprise those selected from among superparamagnetic particles or superparamagnetic particles of two combined magnetizable metals or superparamagnetic particles of oxides of either a single magnetizable metal or two combined magnetizable metals.

30. A process according to claim 23 in which the superparamagnetic particles comprise superparamagnetic particles of an oxide of two combined magnetizable metals, which particles exhibit a spinel structure as determined by X-ray diffraction analysis and Transmission Electron Microscopy.

31. A process according to claim 1 wherein step f is performed instead of step (e).

32. A process according to claim 31 which is repeatedly performed on a series of samples each containing different concentrations of a given biological binding partner, as referred to in step (b) of claim 1, wherein the measurement in step (f) of the intensity of the magnetic signal from the nonpermanent aggregative magnetization of the mass referred to in step (d) of claim 1 is performed uniformly for each sample at the same time interval from the time of removal from the magnetic field of exposure of the mass referred to in each of steps (c) and (d) of claim 1.

33. A process according to claim 31 wherein, once a correlation has been established between the concentration, or number, of the molecules of given biological binding partner, as referred to in step (b) of claim 1, and the intensity of the magnetic signal of the nonpermanent aggregative magnetization of the mass containing it referred to in steps (c) and (d) of claim 1 by measuring said signal in step (f) at a uniform time interval for a series of samples has been obtained as recited in claim 32, the same uniform time interval in step (f) is adhered to whenever the process of claim 31 is performed upon any sample containing an unknown concentration, or number, of molecules of the same biological binding partner.

34. A process according to claim 1 wherein the average mean diameter of the superparamagnetic particles as determined by X-ray diffraction and Transmission Electron Microscopy is in the range of 1 nm to 60 nm.

35. A process for detecting a biological reaction which comprises
(a) conjugating or adsorbing to each of a group of superparamagnetic particles identical biomolecules which are members of a biological binding pair,
(b) contacting the product of step (a) with a sample selected from among liquids and solids, containing or suspected of containing molecules which comprise the biological binding partner of the biomolecules conjugated to or adsorbed on the superparamagnetic particles,
(c) permitting the superparamagnetic particle-biomolecule conjugates or superparamagnetic particle-biomolecule adsorbates from step (a) to react with any biological binding partner molecules present in the aforementioned sample to form a complex, tightly bound, three-dimensional mass comprising interlinked biomolecules and bound superparamagnetic particles;
(d) exposing the said mass to a magnetic field for the shortest period necessary to induce magnetization of the superparamagnetic particles in said mass and then immediately removing the magnetic field and
(e) measuring the intensity of the magnetic signal of said mass with a suitable instrument at, intervals over a period to detect the period of its decay.

36. A process according to claim 35 wherein the product of step (a) is supported on one end of an immunochromatographic (“ICT”) strip, which strip is provided at its other end with a capture zone consisting of immobilized unbound biomolecules identical to those adsorbed in step (a) to superparamagnetic particles, and said product is contacted in step (b) with a liquid sample suspected of containing the biological binding partner for the biomolecules of step (a), which liquid sample picks up the product of step (a) and flows with it along said strip to said capture zone wherein a complex, tightly bound three-dimensional mass of superparamagnetic particle-tagged biomolecule-biological binding partner-immobilized biomolecule is formed, in step (c) and the portion of the ICT strip containing said three-dimensional mass is cut from the strip and maintained in a wet state while being subjected to steps (d) and (e) of claim 35.

37. A process for detecting a biological reaction which comprises
(a) conjugating or absorbing to each of a group of superparamagnetic particles, identical biomolecules which are members of a biological bind pair and depositing the resulting conjugates at a position near the sample-receiving end of an immunochromatographic (“ICT”) strip in a state such that, upon contact with a liquid sample flowing chromatographically along said strip, they will be picked up and will flow along said strip with said liquid sample,
(b) adding a liquid sample known to contain or suspected of containing a biological binding partner of the identical biomolecules referred to in step (a) to the sample receiving end of the ICT strip, allowing said sample to flow chromatographically along the said strip and pick up the movably deposited conjugates formed in step (a) allowing the said sample and said conjugates to flow together along said strip and to form further conjugates of superparamagnetic particle-biomolecule-biological binding partner as the flow proceeds,
(c) allowing the flowing mass of step(b) to contact a previously deposited stripe of an immobilized biomolecule known to be reactive with said biological bonding partner whereupon a complex, tightly bound three dimensional mass comprising interlinked biomolecule-biological binding partner reaction products and tightly bound superparamagnetic particles forms along said stripe,
(d) separating the segment of the ICT strip containing the stripe along which said tightly bound three dimensional mass has formed from the balance of said strip while wet, by cutting or other suitable mechanical means,
(e) maintaining the separated segment in wet form and subjecting it to the influence of a magnetic field to induce magnetization of said superparamagnetic particles, and
(f) measuring the aggregative permanent magnetization of said particles over a desired interval or series of intervals while maintaining the strip in wet condition under the influence of the same magnetic field while holding said magnetic field constant.
